(19) United States
(12) Patent Application Publication
(10) Pub. No.: US 2026/0227354 A1
(43) Pub. Date: Aug. 6, 2026

(54) DEVICE FOR TOMOGRAPHY MEASUREMENT USING A RATIOMETRIC ELECTRICAL PROCESS FOR A SOLID SUBSTRATE

(71) Applicant: TOUCH SENSITY, Pessac (FR)

(72) Inventors: Philippe FREITAS, Pessac (FR); Charles PASSET, Pessac (FR)

(73) Assignee: TOUCH SENSITY, Pessac (FR)

(21) Appl. No.: 19/159,175

(22) PCT Filed: Feb. 20, 2024
(86) PCT No.: PCT/FR2024/050220

§ 371 Date: Aug. 22, 2025

(30) Foreign Application Priority Data
Feb. 23, 2023 (FR) 2301682

(51) Int. Cl. G01N 27/02; G01N 27/04

(52) U.S. Cl. CPC G01N 27/026; G01N 27/041; G01N 27/045

(57) ABSTRACT

A device for tomography measurement using a ratiometric electrical process for a solid substrate, comprising sensors, the inputs of which are connected to each other, so that a tomography measurement using a ratiometric electrical process is performed at the output.

The invention relates to the field of monitoring solid substrates, and more generally monitoring the integrity of structures.

To meet the needs of industry, the quality and robustness of the mechanical elements of a system are evaluated and characterised by placing mechanical parts under stress and measuring the behaviour of the part in relation to its structural integrity. Stressing can be done under real conditions, while the part is integrated into the final system, or via one or several test machines, which will exert various successive forces at various points on the part.

The mechanical behaviour of the parts, in particular in terms of extensometry, is measured by a dedicated system comprising one or more sensors. Although several technologies exist, structural monitoring of mechanical elements is still performed by strain gauges. These have the advantage of being highly sensitive and highly accurate. Their implementation and use are now mastered. Measuring systems dedicated to gauges exist, and various mountings, mainly in a gauge bridge, quarter bridge, double bridge or full bridge, make it possible to compensate for the parasitic effects of temperature. The gauges may be in various forms, individually or in the form of rosettes then integrating several gauges.

Strain gauges have at least two major drawbacks: their directivity and the small active area measured. This is because only the extension in the direction of the gauge is measured, and only below the surface covered by the gauge. As a result, when the stress test aims to measure all deformations along several axes of the surface, or worse, when looking to measure a deformation orthogonal to the surface of the part, it is necessary to incorporate a considerable number of gauges and gauge configurations over all surfaces of the mechanical part. This multiplication of gauges results in a large number of connections and excessively long implementation time.

The Applicant develops alternatives using a breakthrough technology in the field of solid substrates: electrical impedance tomography (EIT). In fact its work has made it possible to move this method out of its medical applications and on soft or liquid substrates, to use it in structural integrity monitoring.

In application FR2114482, the Applicant demonstrated the applicability of tomography measurements using an electrical process to solid substrates, in the non-limiting list of the following applications:
man-machine interface (MMI) intended to stimulate the physical interface and measure in return the correlated signals resulting from the stimulation of the physical interface in order to measure any local impedance variation within the interface, and comprising a continuous or variable electrical stimulation source and an analogue-to-digital converter,
MMI based on electrical impedance tomography and intended to stimulate the physical interface and to measure the correlated signals resulting from the stimulation of the physical interface in order to measure any local impedance variation within the interface, and comprising a continuous or variable electrical stimulation source and an analogue-to-digital converter,
MMI based on electrical resistance tomography and aimed at stimulating the physical interface and measuring the correlated signals in return resulting from the stimulation of the physical interface in order to measure any local resistance variation in the interface, and comprising a continuous or variable electrical stimulation source and an analogue-to-digital converter,
MMI based on electrical capacity tomography and aimed at stimulating the physical interface and measuring the correlated signals in return resulting from the stimulation of the physical interface in order to measure any local capacitance variation within the interface, and comprising a variable electrical stimulation source and an analogue-to-digital converter,
structural health monitoring (SHM) system aimed at stimulating a physical specimen and measuring the correlated signals resulting from the stimulation of the physical component in order to measure any local impedance variation within the sample, and comprising a continuous or variable electrical stimulation source and an analogue-to-digital converter,
SHM system based on electrical impedance tomography and aimed at stimulating a physical specimen and measuring in return the correlated signals resulting from the stimulation of the physical component in order to measure any local impedance variation within the specimen, and comprising a continuous or variable electrical stimulation source and an analogue-to-digital converter,
SHM system based on electrical resistance tomography and intended to stimulate a physical specimen and to measure in return the correlated signals resulting from the stimulation of the physical element in order to measure any local resistance variation within the specimen, and comprising a continuous or variable electrical stimulation source and an analogue-to-digital converter,
SHM system based on electrical capacitance tomography and aimed at stimulating a physical specimen and measuring in return the correlated signals resulting from the stimulation of the physical element in order to measure any local capacitance variation within the specimen, and comprising a variable electrical stimulation source and an analogue-to-digital converter,
non-destructive testing (NDT) system aimed at stimulating a physical specimen and measuring in return the correlated signals resulting from the stimulation of the physical component in order to measure any local impedance variation within the sample, and comprising a continuous or variable electrical stimulation source and an analogue-to-digital converter,
NDT system based on electrical impedance tomography and aimed at stimulating a physical specimen and measuring in return the correlated signals resulting from the stimulation of the physical component in order to measure any local impedance variation within the specimen, and comprising a continuous or variable electrical stimulation source and an analogue-to-digital converter,
NDT system based on electrical resistance tomography and aimed at stimulating a physical specimen and measure in return the correlated signals resulting from the stimulation of the physical element in order to measure any local resistance variation within the specimen, and comprising a continuous or variable electrical stimulation source and an analogue-to-digital converter, and
NDT system based on electrical capacity tomography and aimed at stimulating a physical specimen and measuring in return the correlated signals resulting from the stimulation of the physical element in order to measure any local capacitance variation within the specimen, and comprising a variable electrical stimulation source and an analogue-to-digital converter.

Electrical impedance tomography makes it possible to reduce the number of connections required to perform monitoring. This is because measurements made on a limited number of electrodes placed at the periphery of an active pickup surface and algorithms for reconstruction of the structural characteristics of the pickup surface, allow EIT-based processes to monitor a complete surface, much larger than that captured by a gauge. They also potentially make it possible to determine the extension and deformation components along all axes of the space by visualising the localised stressed zones.

On the other hand, EIT-based processes do not allow one or more active surfaces to be mounted in a bridge as for gauges inherently. Measurements may therefore be sensitive to certain variations such as temperature. On the other hand, EIT will be all the more accurate as the measurements made are accurate and free of noise and variations outside those of the active surface.

Existing solutions, such as those of application FR2114482, allow quality measurements to be performed on one EIT-based sensor, but not on several sensors simultaneously. There is currently no solution for pooling such sensors.

The invention improves the situation. For this purpose, it proposes a tomography measuring device using an electrical process for a solid substrate, comprising a current or voltage source, a reference sensor, at least one other sensor, and at least one comparator arranged to output a signal from the reference sensor or the at least one other sensor, each at least one other sensor comprising a current input, a current output, and a plurality of electrodes suitable for being connected to a solid substrate to be measured, and two measurement inputs of the voltage difference at the terminals of said measurement inputs, each at least one other sensor being capable of performing a measurement sequence within which the current input and the current output are each connected to a respective electrode of a pair of excitation electrodes of the plurality of electrodes, and the two measurement inputs are each connected to a respective electrode of a pair of measurement electrodes of the plurality of electrodes, the pair of excitation electrodes and the pair of measurement electrodes being defined by the measurement sequence at each measurement and the pair of measurement electrodes being connected to the at least one comparator to measure the propagation in the solid substrate of the current introduced via the pair of excitation electrodes. The current source is connected to the reference sensor, which comprises a current output connected to the current input of the at least one other sensor and is connected to the at least one comparator to output a signal corresponding to a voltage difference according to the measurement sequence. The device is further arranged to determine the ratio between the voltage difference at the terminals of the measurement inputs of each at least one other sensor for each measurement sequence and the voltage difference at the terminals of the measurement inputs of the reference sensor.

This device is particularly advantageous because it makes it possible to use architectures based on the pooling of the stimulation of active surfaces and on the principle of ratiometric measurement between multiple surfaces, for structural monitoring systems based on multi-element comparative electrical tomography.

According to various embodiments, the invention may have one or more of the following characteristics:
each sensor comprises a multiplexer connected on the one hand to its current input and to its current output and on the other hand to its plurality of electrodes,
each sensor comprises two multiplexers connected on the one hand to its plurality of electrodes, and on the other hand to the two measurement inputs of the at least one comparator,
each at least one other sensor comprises a comparator connected to the two measurement inputs,
the reference sensor comprises a current input, a current output, a plurality of electrodes suitable for being connected to a solid substrate to be measured and a comparator comprising two measurement inputs arranged to emit a signal corresponding to the voltage difference at the terminals of said measurement inputs, the reference sensor being capable of performing a measurement sequence within which the current input and the current output are each connected to a respective electrode of a pair of excitation electrodes of the plurality of electrodes, and the two measurement inputs of the comparator are each connected to a respective electrode of a pair of measurement electrodes of the plurality of electrodes, the pair of excitation electrodes and the pair of measurement electrodes being defined by the sequence of measurements at each measurement and the propagation in the solid substrate to be measured of the current introduced via the pair of excitation electrodes being measured at the terminals of the pair of measurement electrodes,
the comparator of the reference sensor is a differential amplifier, and wherein the comparator of the sensors is an analogue-to-digital converter further comprising a reference input connected to the output of said differential amplifier and arranged to emit a digital signal corresponding to the difference between the voltage of the two measurement inputs proportionally to a voltage level designated by the reference input,
wherein each comparator is an analogue-to-digital converter further comprising a reference input arranged digital signal to output a corresponding to the difference between the voltage of the two measurement inputs proportionally to a voltage level designated by the reference input, the device further comprising a divider arranged to calculate the ratio between the digital signal of each at least one other sensor and the digital signal of the reference sensor,
the reference sensor is a control sensor, and
the reference sensor is a calibration specimen reproducing the expected voltage differences for a solid substrate to be measured.

The invention also relates to a solid substrate, characterised in that it comprises a device as described previously, added on at least one of the surfaces or integrated.

Other features and advantages of the invention will become apparent upon reading the following description, with reference to examples provided for illustrative and non-limiting purposes, with reference to the drawings wherein:

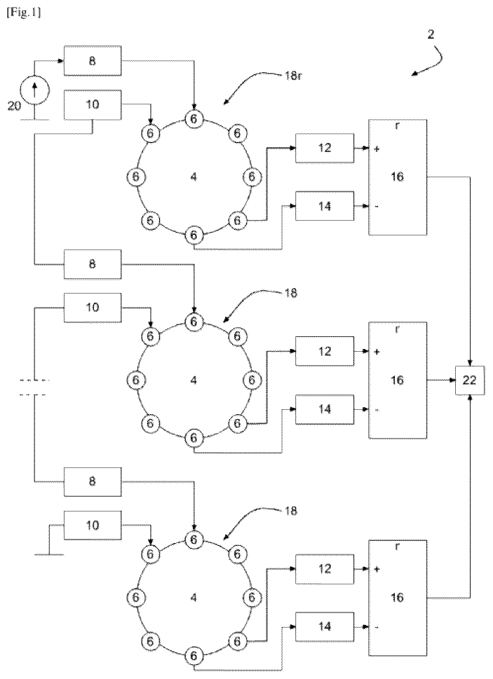

FIG. 1 shows a diagram of a first embodiment of a device according to the invention,

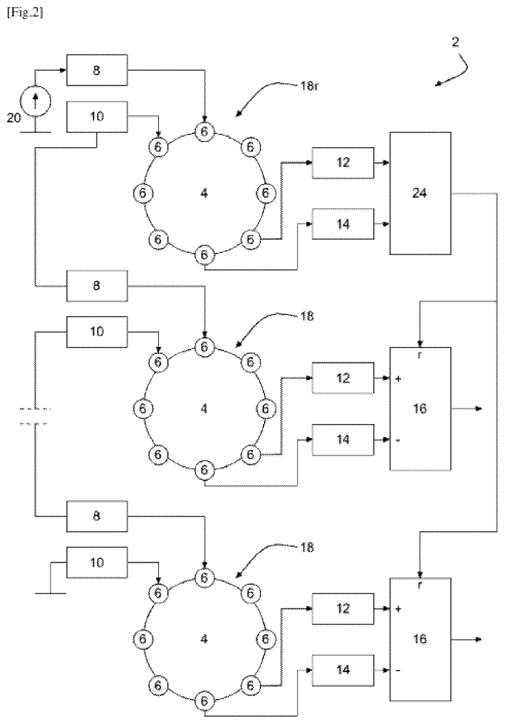

FIG. 2 shows a diagram of a second embodiment of the device according to the invention

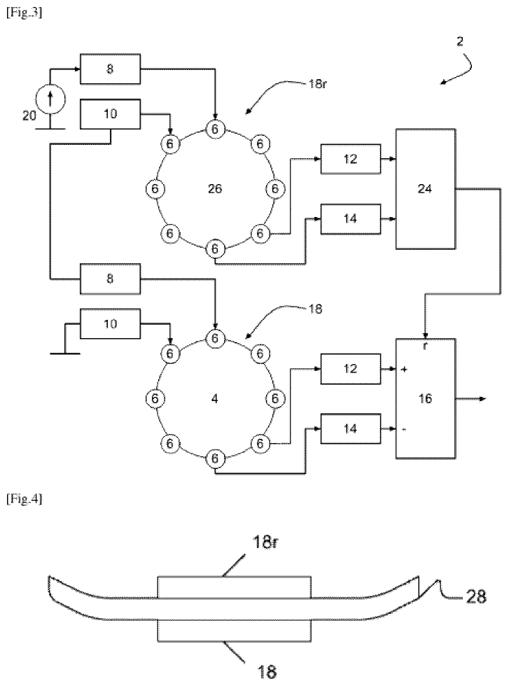

FIG. 3 shows a diagram of a third embodiment of a device according to the invention,

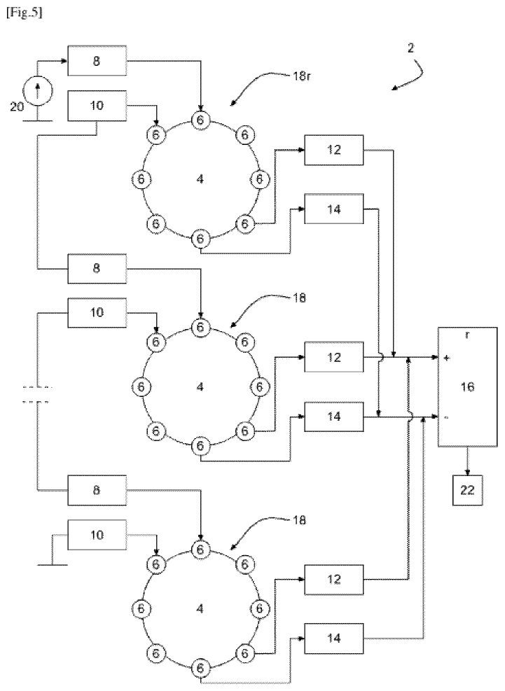

FIG. 4 shows a particular implementation of the embodiment in FIG. 3, and

FIG. 5 shows a diagram of a fourth embodiment of a device according to the invention.

The drawings and the description below contain, for the most part, elements of a certain nature. They may therefore not only serve to improve understanding of the present invention, but also contribute to its definition, where appropriate.

The architectures of EIT sensors as described in application FR2114482 have the following general structure: a current or voltage source supplies a first multiplexer which is connected to a plurality of electrodes. This assembly makes it possible to produce an excitation sequence by which a current is successively introduced between pairs of electrodes, neighbouring or distant, according to a chosen excitation pattern. These excitations are measured by pairs of electrodes via a second multiplexer which connects the electrodes to the input of an analogue to digital converter which makes it possible to recover the measurement of the voltage that has propagated in the substrate.

All the measurements can then be processed in order to reconstruct the electrical field of a conductive part of the substrate and to derive information from it on the state or changes thereof.

When wishing to use a plurality of EIT sensors to qualify a structure, it is possible to use an architecture where the sensor measurements are carried out in series, or in parallel.

In the case of the serial architecture, the stimulation source and the acquisition assembly are common to all the measurements performed on all the specimens. These are switched on the various specimens via additional multiplexers upstream and downstream of the multiplexers specific to each specimen. In this case, each measurement is taken at a given time and no others are taken at the same time. All measurements are therefore decorrelated. Noise and variations, especially from the stimulation source, cannot be easily filtered out. These fluctuations will be detrimental to the performance of the reconstruction algorithms.

In the case of the parallel architecture, a plurality of current sources and acquisition assemblies are used. This system is faster but more expensive. Similarly, and even if the parallel measurements are carried out at the same time on the various specimens, as the various current sources and acquisition assemblies are not correlated, their respective noise and variations will not be correlated either.

The system of FIG. 1 shows a device 2 which makes it possible to obtain measurement speeds of the type of the architecture in parallel with a cost structure close to that of the serial architecture. In addition, it offers much better accuracy and noise performance than the previous two architectures thanks to the pooling of stimulation for all sensors. The effects of noise and stimulation variations are then correlated across all measurements and therefore more easily filtered out.

For this purpose, the device of FIG. 1 comprises a plurality of specimens 4 on each of which a plurality of electrodes 6 are disposed. The device 2 also comprises pairs of multiplexers 8 and 10 associated with each plurality of electrodes 6 in order to perform the current injection. More particularly, the multiplexer 8 serves to inject the current, while the multiplexer 10 serves to recover the injected current. Thus the multiplexer 8 may be seen as a current input and the multiplexer 10 as a current output.

The device 2 also comprises pairs of multiplexers 12 and 14 associated with each plurality of electrodes 6 in order to perform the measurement. Each pair of multiplexers 12 and 14 is connected to respective measurement inputs of a comparator 16 that performs the voltage measurement for the EIT for the specimen 4 concerned. The multiplexers 12 and 14 can be seen as the measurement inputs of the comparators 16.

The plurality of electrodes 6, the multiplexers 8, 10, 12, 14 and the comparator 16 together form a respective sensor 18 for each specimen 4.

The device further comprises a current source 20 which is connected to the multiplexers 12 and 14 of one of the sensors 18. This sensor will hereinafter be called the reference sensor and referenced 18r hereinafter because it serves as the basis for ratiometric measurement. In the rest of the device 2, the multiplexer 10 is connected to the multiplexer 8 of another sensor 18, except the multiplexer 10 of the last sensor 18, which is connected to ground.

The measurements in this EIT architecture are all dependent on the same source, at all times. The device 2 therefore makes it possible to avoid noise and relative variations from the sources of the conventional parallel multi-element structure. Connecting the specimens to each other is relatively simple due to the connection of the input of the specimen #n to the stimulation output of the specimen #n−1.

In the case of voltage stimulation, the multiplexer inputs must be interconnected in parallel. Thus, if a variation or noise appears on the stimulation source, all measurements will be affected by this variation. Filtering of this variation in the data will then be facilitated downstream.

In the embodiment in FIG. 1, all the comparators 16 are analogue-to-digital converters, the outputs of which are connected to a divider 22 such that the latter divides each sensor output 18 by the output of the reference sensor 18r. Thus the device 2 performs a ratiometric measurement of all the sensors 18 with respect to the reference sensor 18r. The divider 22 may be implemented in any manner allowing a ratio to be achieved between two digital inputs.

The information measured by the device 2 is therefore not found in the absolute measurements for each specimen 4 but in the ratio between the measurements obtained on each specimen 4 with respect to the specimen of the reference sensor 18r. This is why it is called a ratiometric measurement.

Considering for example a noisy stimulation current such as Iₛₜᵢₘ=I_{ref}+I_{ε}, then the equivalent measurements on a specimen #1 and #2 will be respectively V₁=α₁ (I_{ref}+I_{ε}) and V₂=α₂ (I_{ref}+I_{ε}), with α₁, α₂ the conversion ratios between measured values and stimulation current. The corresponding ratiometric measurement with, for example, specimen #1 as reference, will then be

$$\frac{{V}_{2}}{{V}_{1}} = \frac{{α}_{2}}{{α}_{1}} .$$

Therefore, the noise from the source disappears from the equation. The same would apply to absolute variations in the current source, noise picked up on all specimens, or temperature effects on specimens.

FIG. 2 shows an alternative embodiment in which the ratiometric measurement is performed without a divider. Thus the comparator 16 of the reference sensor 18r is here a differential amplifier 24 whose output is an analogue signal which is a multiple of the voltage difference of the measurement inputs. This analogue signal is used as a reference voltage transmitted to the reference voltage input r of all comparators 16 of the other sensors 18, which are analogue-to-digital converters. Thus the output of each comparator 16 is inherently a ratiometric measurement between each sensor 18 and the reference sensor 18r.

The device in FIGS. 1 and 2 can be used to replace the strain gauges in the SHM. As mentioned above, strain gauges are often mounted in pairs or in larger combinations of gauges, to compensate for potential variabilities external to the measurement such as temperature. These arrangements can be replicated using capture surfaces (patches) measured using EIT measurement sensors. These capture surfaces are then stuck onto the structure to be analysed and coupled together via the device 2, whose ratiometric measurement makes it possible to obtain an independent measurement of external variability. This optimises the ratiometric measurement by removing the variability due to stimulation of the measurements.

FIG. 3 again shows a variant embodiment of FIG. 2. In this embodiment, there are only two sensors in total, and the reference sensor 18r is a control or a standard 26. This control may be a model of the measurement sought or even a “phantom” (see for example the article by H Griffiths, (1988), “A phantom for electrical impedance tomography”, Clin Phys Physiol Meas., 9 Suppl A: 15-20), i.e. an electronic circuit arranged to react as a control specimen.

The sensor 18r was previously characterised, potentially by other more precise but also more expensive and/or slower test means. Once available, the data from this specimen is compared with the measurements from a specimen at the end of production. This principle implies that the measurements are carried out by the same device 2 and that any variability other than that specific to the specimen under test is minimised. In the context of the comparison with a control specimen on a production monitoring system based on the EIT, the invention offers the advantage of cancelling the variability of the measurement parameters between the two specimens.

FIG. 4 shows an example implementation of the embodiment of FIG. 3 in the context of an MMI, wherein the reference sensor 18r and another sensor 18 for ratiometric measurement are placed on either side of a surface 28 of a substrate. Thus the device 2 makes it possible to detect the pressure on a surface of the substrate since the sensors 18r and 18 will deform differently.

FIG. 5 represents yet another embodiment. In this embodiment, not only the current source 20 has been pooled, but the comparator 16 has also been pooled, so that there is a single analogue-to-digital converter for the entire device 2. In this embodiment, the measurements must therefore be multiplexed, i.e. a measurement of each measurement sequence is performed sequentially on the sensor 18r and on each sensor 18 before proceeding to the next measurement of the measurement sequence.

This embodiment has the advantage of being the most economical in hardware, and has the disadvantage of having to multiplex operations, which can cause a time consistency problem when there are many sensors 18.

In the foregoing, all or part of the device 2 may be added to at least one of the surfaces by glueing, deposition or any other attachment or integrated into the solid substrate. By integrated with the solid substrate, it should that be understood the solid substrate can be composed of several layers, such as sandwich composites or the like, and the sensors can be integrated therein, in particular when one or more of them are conductive. Furthermore, by solid substrate, any substrate in the solid state should be understood, whether it is rigid or has a certain elasticity.

1. A tomography measurement device using an electrical process for a solid substrate, comprising a current or voltage source, a reference sensor, at least one other sensor, and at least one comparator configured to emit a signal from the reference sensor and/or the at least one other sensor,
each at least one other sensor comprising a current input, a current output, and a plurality of electrodes suitable for being connected to a solid substrate to be measured, and two measurement inputs of the voltage difference at the terminals of said measurement inputs,
each at least one other sensor being configured for performing a measurement sequence within which the current input and the current output being each connected to a respective electrode of a pair of excitation electrodes of the plurality of electrodes, and the two measurement inputs being each connected to a respective electrode of a pair of measurement electrodes of the plurality of electrodes, the pair of excitation electrodes and the pair of measurement electrodes being defined by the measurement sequence at each measurement and the pair of measurement electrodes being connected to the at least one comparator to measure the propagation in the solid substrate of the current introduced via the pair of excitation electrodes,
the current source being connected to the reference sensor, which comprises a current output connected to the current input of the at least one other sensor and is connected to the at least one comparator to output a signal corresponding to a voltage difference according to the measurement sequence,
the device being further configured to determine the ratio between the voltage difference at the terminals of the measurement inputs of each at least one other sensor for each measurement sequence and the voltage difference at the terminals of the measurement inputs of the reference sensor.

2. The tomography measurement device according to claim 1, wherein each sensor comprises a multiplexer connected on the one hand to its current input and to its current output, and on the other hand to its plurality of electrodes.

3. The tomography measurement device according to claim 1, wherein each sensor comprises two multiplexers connected on the one hand to its plurality of electrodes, and on the other hand to the two measurement inputs of the at least one comparator.

4. The tomography measurement device according to claim 3, wherein each at least one other sensor comprises a comparator connected to the two measurement inputs.

5. The tomography measurement device according to claim 1, wherein the reference sensor comprises a current input, a current output, a plurality of electrodes suitable for being connected to a solid substrate to be measured and a comparator comprising two measurement inputs configured to emit a signal corresponding to the voltage difference at the terminals of said measurement inputs, the reference sensor being configured for performing a measurement sequence within which the current input and the current output being each connected to a respective electrode of a pair of excitation electrodes of the plurality of electrodes, and the two measurement inputs of the comparator being each connected to a respective electrode of a pair of measurement electrodes of the plurality of electrodes, the pair of excitation electrodes and the pair of measurement electrodes being defined by the sequence of measurements at each measurement and the propagation in the solid substrate to be measured of the current introduced via the pair of excitation electrodes being measured at the terminals of the pair of measurement electrodes.

6. The tomography measurement device according to claim 5, wherein the comparator of the reference sensor is a differential amplifier, and wherein the comparator of the sensors is an analogue-to-digital converter further comprising a reference input connected to the output of said differential amplifier and configured to emit a digital signal corresponding to the difference between the voltage of the two measurement inputs proportionally to a voltage level designated by the reference input.

7. The tomography measurement device according to claim 1, wherein each comparator is a digital analogue converter further comprising a reference input configured to emit a digital signal corresponding to the difference between the voltage of the two measurement inputs proportionally to a voltage level designated by the reference input, the device further comprising a divider configured to calculate the ratio between the digital signal of each at least one other sensor and the digital signal of the reference sensor.

8. The tomography measurement device according to claim 1, wherein the reference sensor is a control sensor.

9. The tomography measurement device according to claim 1, wherein the reference sensor is a calibration specimen reproducing expected voltage differences for a solid substrate to be measured.

10. A solid substrate, comprising the tomography measurement device according to claim 1, added on at least one surface or integrated.
